SEQUENCE LISTING (12 sequences)

SEQ ID NO: 1 - DNA, 26 bp, Homo sapiens
ctccatgatgcgttatctgggtctgg

SEQ ID NO: 2 - DNA, 26 bp, Homo sapiens
cctatggcgcccctgctgggccactg

SEQ ID NO: 3 - protein, 18 aa, Homo sapiens
  misc_feature (17)..(17): Xaa can be any naturally occurring amino acid
QAPPGAYPGQAPPGAYXG

SEQ ID NO: 4 - protein, 18 aa, Macaca mulatta
QAPPGVYPGQAPPGAYPG

SEQ ID NO: 5 - protein, 19 aa, Canis lupus
QAPPGGYPGQAPPGGYPGQ

SEQ ID NO: 6 - protein, 18 aa, Bos primigenius
QAPPGPYPGGPPGAYPGQ

SEQ ID NO: 7 - protein, 19 aa, sus
QGPPGAYPGQAPPGAYPGQ

SEQ ID NO: 8 - protein, 16 aa, Oryctolagus cuniculus
APGAYPGQAPPGPYPG

SEQ ID NO: 9 - protein, 19 aa, Mus musculus
QAPPGAYPGQAPPSAYPGP

SEQ ID NO: 10 - protein, 19 aa, Rattus rattus
QAPPGGYPGQAPPSAYPGP

SEQ ID NO: 11 - protein, 19 aa, Phodopus campbelli
QAPPGAYPGQAPPGAYPGP

SEQ ID NO: 12 - protein, 16 aa, Gallus gallus
GPPGPYPGGPPGPYPG

(19) United States
(12) Patent Application Publication
(10) Pub. No.: US 2012/0095117 A1
(43) Pub. Date: Apr. 19, 2012

(54) GALECTIN-3 nsSNP MARKER FOR CANCER

(75) Inventors: Avraham Raz, West Bloomfield, MI (US); Vitaly Balan, Troy, MI (US); Pratima Nangia-Makker, Troy, MI (US)

(73) Assignee: Wayne State University, Detroit, MI (US)

(21) Appl. No.: 13/324,184

(22) Filed: Dec. 13, 2011

Related U.S. Application Data
(62) Division of application No. 12/365,588, filed on Feb. 4, 2009, now abandoned.

(51) Int. Cl. A61K 45/00; G01N 33/574; C12Q 1/68

(52) U.S. Cl. 514/789; 435/6.11; 435/7.92; 436/501

(57) ABSTRACT

A diagnostic biomarker including a mechanism for determining a patient's propensity to develop cancer. A diagnostic kit for determining a patient's propensity to develop cancer. A method of predicting both a patient's and a population's propensity to develop cancer, by detecting the presence of an H⁶⁴ and/or P⁹⁸ allele of galectin-3 from a patient's serum sample, and predicting that the patient or population has the propensity to develop cancer based on the presence of the allele. A method of providing prophylactic cancer treatment, by detecting the presence of an H⁶⁴ and/or P⁹⁸ allele of galectin-3 from a patient's serum sample, predicting that the patient has the propensity to develop cancer based on the presence of the allele, and recommending and providing prophylactic treatment to the patient to reduce their risk of cancer. A prophylactic anticancer treatment and method of preventing onset or further proliferation of cancer.

### GOVERNMENT GRANT INFORMATION

Research in this application was supported in part by a grant from the National Institute Of Health (NIH Grant No. 2R37CA46120-19). The Government has certain rights in the invention.

### BACKGROUND OF THE INVENTION

1. Field of the Invention

The present invention relates to markers, diagnostic kits, therapeutic compounds and Methods for the detection of cancer. In particular, the present invention relates to the marker galectin-3 nsSNP rs4644 and nsSNP rs4652 as markers for cancer.

2. Description of Related Art

Breast cancer is one of the most common causes of cancer death among women. It is estimated that in 2008 about 182,460 new cases of invasive breast cancer will be diagnosed among women in the United States. Although many epidemiological and etiological risk factors have been identified, the cause of any individual breast cancer is often unknown. Breast cancer incidents and mortality can vary tremendously with race.

To date, numerous attempts have been made at identifying potential breast cancer risk alleles and genetic signatures and their distribution across women populations. Two major breast cancer hereditary susceptibility genes BRCA1 and BRCA2 have been discovered. These genes are involved in the repair of damaged DNA. Mutations in BRCA1 and BRCA2 prohibit correct DNA repair, and thus defective cells accumulate and progress into cancer. While women having mutations of these genes and have a family history of the disease are much more at risk to develop breast and other cancers, mutations of BRCA1 and BRCA2 are responsible for only ˜5% of all breast cancer incidents. No other obvious candidate genes contributing to a significant breast cancer risk have been identified.

Mutations in p53, ATM, Chek2, Ras, Her2, CCND1 and other gene products over-expression and ESR1 amplification were implicated for a small fraction of breast cancer cases. Some of these mutations were associated with more aggressive disease and a worse overall survival rate. However, with only a relatively small percent of the population with breast cancer having mutations in any of these genes, they are not considered a suitable marker for diagnosing breast cancer.

A number of recent reports have focused on single nucleotide polymorphisms (SNP) with the notion that such a genetic event can possibly contribute to breast cancer development as well as other cancers. Using genomic-wide association studies (GWAS) and intensive bioinformatic searches of existing databases, it was demonstrated that non-synonymous (ns) SNPs of a few candidate genes have very limited association with breast cancer. Some of these studies were restricted/focused only on already established oncogenes or genes of perceived interest and many other genes of potential significance were overlooked. While a powerful experimental tool, GWAS are not without challenges; critical to success is the development of robust study designs, sufficient sample sizes, rigorous phenotypes and comprehensive maps.

Candidate gene studies conducted to date have focused mainly on established oncogenes or genes of perceived interest while many other genes of potential significance have been overlooked. For example, the commercially available Affymetrix Genome-Wide Human SNP Array 6.0 features 1.8 million genetic markers, includes more than 906,600 SNPs while still missing numerous others. The Human Hap550 chip from Illumina suffers from a similar shortcoming.

The present invention reports two nsSNP rs4644 and rs4652 in the galectin-3 gene that have not previously been evaluated. Galectin-3 is a member of the galectin gene family of animal lectins expressing binding/specificity to β-galactoside residues through evolutionarily conserved sequence elements of a carbohydrate recognition/binding domain (CRD). Galectin-3 is an evolutionary conserved chimeric gene product consisting of a short NH2-terminal domain of 12 amino acids that contains a serine phosphorylation site that regulates its cellular targeting, a collagen-like repeated sequence of about 110 amino acids rich in glycine, tyrosine and proline residues, serving as a substrate for matrix metalloproteinases (MMPs), and a COOH-terminal domain of about 130 amino acids that contains a single CRD and the NWGR anti-death motif of the Bcl2 gene family.

Clinical investigations have shown a correlation between expression of galectin-3 and the malignant properties of several types of cancer; consequently, galectin-3 is thought to be a cancer-associated protein. An allelic variation (C to A) in the DNA sequence of galectin-3 at position 191 (rs4644), which substitutes the proline (P) 64 to histidine (H), was previously noted and, importantly, regarded as non-functional mutation. Recently, applicants have addressed the possible function and significance of this mutation. Amino acids Ala62-Tyr63 of galectin-3 harbor the actual cleavage site for MMP-2 and -9 and substitution of the subsequent H64 with P resulted in loss susceptibility to cleavage by MMPs (Cancer Res. In press). Transfection of galectin-3 containing H64 resulted in tumorigenic acquisition of breast cancer and colon cancer cells in a xenograft mouse model. In sharp contrast to the BT-549 cells transfected with the oncogenic variant of galectin-3 e.g., galectin-3H64, cells transfected with galectin-3P64 showed reduced tumorigenesis associated with reduced angiogenesis and increased apoptosis suggesting that the extracellular cleavage of secreted galectin-3H64 by MMPs plays a significant role during tumor development/progression (Cancer Res. In press). Another allelic variation that can be risk factor based on data herein is rs4652, which substitutes the threonin (T) 98 to proline (P).

However, there has never been a showing of the significance of the presence of the H64 versus the P64 and T98 vs P98 in galectin-3, i.e. whether these SNPs are risk factors for cancer, especially in human studies. Knowledge of whether these SNPs are risk factors would aid in diagnosing patients while treatment can still be obtained to eradicate the cancer, or even allow the patient to take steps to prevent cancer from occurring in the first place. While cancer detection and treatment has progressed over the years, in many instances individuals do not show any symptoms of cancer until treatment is futile.

Based on the available data, it is obvious that breast cancer is a complex disease, consisting of diverse structures, genetic and genomic variations, and clinical outcomes [28]. Genetic linkage studies failed to add a significant number of breast cancer-causing genes following the heredity association of BRCA1 and BRCA2 genes' mutation to breast cancer [29]. Since SNPs may contribute to the development of diseases it has been suggested that breast cancer susceptibility is conferred by a large number of loci, each contributing a small additive to the overall breast cancer risk [4]. Of note, except for BRCA1/2, to date no global risk pattern for breast cancer like diet, local environmental factors and/or genetic predisposition has been identified. Further, despite the recognition of multiple genetic and environmental risk factors for breast cancers, development of the majority of clinical cases lack an identifiable risk factor(s) other than age, race and gender.

Thus, there remains a need for methods of detecting cancer and propensity to develop cancer based on genetics of a patient, both for breast cancer and other types of cancers. While the biochemistry of galectin-3 has previously been studied, there has never before been any indication of a mutation or SNP that is universally found in cancer patients. Therefore, there is a need for studies to determine the significance of the SNPs in galectin-3.

BRIEF SUMMARY OF THE INVENTION

The present invention provides for a diagnostic biomarker including a mechanism for determining a patient's propensity to develop cancer.

The present invention also provides for a diagnostic kit for determining a patient's propensity to develop cancer, including an assay to detect the presence of an H64 and/or P98 allele of galectin-3 from a patient's serum sample.

The present invention further provides for a method of predicting propensity to develop cancer, including the steps of detecting the presence of an H64 and/or P98 allele of galectin-3 from a patient's serum sample, and predicting that the patient has the propensity to develop cancer based on the presence of the allele.

The present invention provides for a method of predicting a population's propensity to develop cancer, including the steps of detecting the presence of an H64 and/or P98 allele of galectin-3 from serum samples of patients in a population, and predicting the percentage of the population that has the propensity to develop cancer based on the presence of the allele.

The present invention also provides for a method of providing prophylactic cancer treatment, including the steps of detecting the presence of an H64 and/or P98 allele of galectin-3 from a patient's serum sample, predicting that the patient has the propensity to develop cancer based on the presence of the H64 and/or P98 allele, and recommending and providing prophylactic treatment to the patient to reduce their risk of cancer.

The present invention also provides for a prophylactic anticancer treatment of an effective amount of a compound restricting access to a cleavage site of a cancer associated mutation in galectin-3 in a pharmaceutically acceptable carrier.

Finally, the present invention provides for a method of preventing the onset or further proliferation of cancer, including the steps of detecting the presence of an H64 allele of galectin-3 from a patient's serum sample, predicting that the patient has the propensity to develop cancer based on the presence of the H64 allele, administering the prophylactic anticancer treatment described above to the patient, and restricting access to an Ala62-Tyr63 cleavage site of H64 allele of galectin-3 in the patient.

### BRIEF DESCRIPTION OF THE SEVERAL VIEWS OF THE DRAWINGS

Other advantages of the present invention will be readily appreciated as the same becomes better understood by reference to the following detailed description when considered in connection with the accompanying drawings wherein:

FIG. 1A is a table of distribution of nsSNP rs4644 among cell lines and their tumorigenicity and FIG. 1B is a multiple sequence alignment for galectin-3 of chicken and a few mammalian species (dots in alignment represent gaps, grey boxes show the identity between the proteins, X represents either H or P, and the numbers represent the position of amino acids in human galectin-3).

FIG. 2A is a graph of genotype distribution of rs4644 from 144 cancer-free Caucasian and 20 Asian volunteer women and FIG. 2B is a graph of genotype distribution data from 50 Caucasian and 59 Asian unrelated women from Ensembl release 46 (Black bar: H—homozygous alleles; lined bar: H/P—heterozygous alleles; open bar: P—homozygous alleles), and the inset of FIG. 2B shows breast cancer incidence by race (rates expressed as cases per 100,000, statistics were generated from malignant cases only).

FIG. 3A is a graph of genotype distribution of nsSNP from 141 Caucasian and 61 Asian breast cancer patients determined using independent sequencing and RFLP analysis, and FIG. 3B is a RFLP and sequence analysis of the C191A site of the galectin-3 gene (insets: DNA sequence chromotograms indicating allelic variation).

FIGS. 4I and 4II show cisplatin induced apoptosis of BT-549 cells and cloned variants, FIG. 4I: % apoptosis in cells treated with 25 uM cisplatin for 72 hr at 370 C and analyzed following MTT assay (ODs in the untreated control cells were calculated as 100%, and the values are the average of triplicate experiments, bars represent ±SD, *P>0.001), FIG. 4II: Western blot analysis of the expression of PARP in cells treated with 50 μM cisplatin after 24, 48, and 72 hours, IIA: BT-549; IIB: BT-549 Gal-3 H64; IIC: BT-549 Gal-3 P64 (β-Actin was used as loading control. 1—untreated; 2—24 hours; 3—48 hours; 4—72 hours), FIGS. 4IIIA-B and 4IIIA′-C′: Immunohistochemical analysis of intact and cleaved galectin-3 in breast cancer progression.

### DETAILED DESCRIPTION OF THE INVENTION

The present invention provides for, generally, diagnostic biomarkers, diagnostic kits, and methods of predicting a patient's propensity to develop cancer by determining the presence of nsSNPs of galectin-3.

The term “biomarker” as used herein refers to a substance, such as, but not limited to, a protein, DNA sequence, RNA sequence, or other biological sequence that, when detected, indicates a particular disease state or propensity to develop a particular disease state. The biomarker can be detected in a patient to diagnose disease or diagnosis propensity to develop disease. The biomarker can also be used to personalize therapeutic treatment.

The term “diagnostic” as used herein refers to a test or method to identify a medical condition or disease state, such as propensity to develop cancer.

The term “cancer” as used herein refers to any disease in which cells aggressively grow and divide with respect to normal cells, invade and destroy adjacent tissues, and/or metastasize and spread to one or more areas of the body. While the data provided herein is related to breast cancer (and several different types of breast cancer), the biomarkers and methods of the present invention can be used with any other type of cancer.

The term “prophylactic” as used herein refers to any procedure that is performed to prevent disease, or prevent worsening of disease. Prophylactic methods can include, but are not limited to, surgical procedures and/or drug treatment.

The term “serum” as used herein refers to any biological fluid that can be extracted from a patient and can be analyzed for the content of DNA, mRNA (also referred to as RNA), protein, and/or any other nucleotide material of interest.

The present invention provides for a diagnostic biomarker for determining a patient's propensity to develop cancer, wherein the biomarker is a cancer associated mutation of galectin-3. More specifically, this biomarker is the presence of an H64 and/or P98 allele of galectin-3. As further described below, the H64 and P98 allele is found to be associated with more than 97% and about 95% respectively of breast cancer cases in the studies performed herein. The H64 allele can either be homozygous (i.e. H/H or P/P) or heterozygous with proline (i.e. H/P). The P98 allele can either be homozygous (i.e. P/P or T/T) or heterozygous with threonine (i.e. T/P). While the homozygous H64 and/or P98 alleles are found in most cancer cases, the heterozygous allele also contributes to the propensity to develop cancer. The presence of either of these alleles, with or without other factors, greatly increases the chances that a patient will develop cancer. Therefore, the presence of the homozygous P64 and/or P98 allele indicates a lower propensity to develop cancer. The diagnostic biomarker is useful in determining a patient's propensity to develop many different cancers, including, but not limited to, breast cancer. While galectin-3 and the H64 allele were previously known to generally have a role in cancer, there was no indication that the allele was a genomic indicator of the onset of cancer.

The H64 biomarker is the amino acid histidine (H) which arises due to an allelic variation (C to A) in the DNA sequence of galectin-3 at position 191 (rs4644). Without the variation, proline (P) would be at that particular position. Histidine, chemically known as 2-amino-3-(3H-imidazol-4-yl)propanoic acid, has an imidazole side chain with a basic and an acidic side that can contribute to different biological functions. Histidine is coded for with RNA codons CAU and CAC, whereas proline is coded for with CCU, CCC, CCA, and CCG. Thus, the change in CCU of proline to CAU results in the histidine allele.

The P98 biomarker is the amino acid proline (P) which arises due to an allelic variation (A to C) in the DNA sequence of galectin-3 at the position of rs4652. Without the variation, threonine (T) would be at that particular position. Proline, chemically known as (S)-Pyrrolidine-2-carboxylic acid, is an α-amino acid. Proline is coded for with RNA codons CCU, CCC, CCA, and CCG as stated above, and threonine is coded for with ACU and ACA. Thus, a change in ACU or ACA of threonine to CCU or CCA respectively results in the proline allele.

The present invention also provides for a diagnostic kit for determining a patient's propensity to develop cancer, and includes an assay to detect the presence an H64 allele of nsSNP rs4644 and/or a P98 allelle of nsSNP rs4652 of galectin-3 from a patient's serum sample. The kit determines the presence of both homozygous and heterozygous H64 and/or P98 alleles, as each can contribute to the propensity to develop cancer in a patient, alone or with the combination of other risk factors. The kit contains any necessary equipment for obtaining a serum sample from a patient and analyzing the patient's serum, such as, but not limited to, swabs, needles, biological solutions, an immunoassay such as ELISA, buffers, and reagents. The kit can include any detection system that can distinguish between proline and histidine or between proline and threonine at the appropriate position in galectin-3. The kit can analyze DNA, RNA, mRNA, or protein from the patient's serum in order to detect the alleles. The kit can also contain a standard SNP detection system, which can detect SNPs from genomic DNA samples and compare them to control DNA by PCR amplification (for example, ACYCLO-PRIME-FP SNP Detection Systems by PerkinElmer Life Sciences) or detect the SNP from mRNA sequence by RT-PCR and subsequent PCR amplification and sequencing to detect the dominant allele in case of heterozygotes. Thus, a control DNA sample of galectin-3 including the H64 homozygous or heterozygous allele or the P64 homozygous allele can be compared against a patient's serum sample. The same can be compared with the P98 homozygous/heterozygous/T98 homozygous alleles. Kits can be purchased by medical personnel to test patients in a hospital setting, medical clinic, or outpatient setting. In use, a sample of serum is taken from a patient and then applied in the assay along with any other necessary procedures to allow detection of the SNP to take place. Based on the results of the assay, the patient will know whether or not they have a propensity for developing cancer, and can therefore seek prophylactic treatment, as further described below, or change their current behaviors or lifestyle in order to decrease their risk of developing cancer.

The present invention also provides for a method of predicting propensity to develop cancer, including the steps of detecting the presence of an H64 allele and/or a P98 allele of galectin-3 from a patient's serum sample, and predicting that the patient has the propensity to develop cancer based on the presence of the H64 and/or P98 alleles. Either the presence of the homozygous or heterozygous H64 and/or P98 alleles is detected, as either the homozygous or heterozygous allele can indicate the propensity to develop cancer. Again, this method can be used to predict the propensity to develop any cancer, including, but not limited to, breast cancer. Further, the diagnostic kit as described above can be used to detect the presence of the alleles. This method can be performed by any medical personnel in a hospital, medical clinic, or outpatient setting with the diagnostic kit. Samples of the patient's serum are taken and compared to control serum in order to test for the presence of the H64 and/or P98 allele. If the results of the test show that the patient is a carrier of the H64 and/or P98 allele, the medical personnel can inform the patient that they are at risk for developing cancer and prophylactic treatment can be sought as described below.

The present invention further provides for a method of predicting a population's propensity to develop cancer, including the steps of detecting the presence of an H64 and/or P98 allele of galectin-3 from serum samples of patients in a population, and predicting the percentage of the population that has the propensity to develop cancer based on the presence of the H64 and/or P98 allele. This method can be used to determine cancer propensity rates between different populations, such as different races, as described below between Asian and Caucasian races. Such information is useful for tailoring different treatments and disease awareness to different populations. Either the presence of the homozygous or heterozygous H64 and/or P98 allele is detected, as either the homozygous or heterozygous allele can indicate the propensity to develop cancer. The presence of either allele can be additive to gain the percentage of the population with a propensity for developing cancer. Again, this method can be used to predict the propensity to develop any cancer, including, but not limited to, breast cancer. Further, the diagnostic kit as described above can be used to detect the presence of the alleles. A sample size of a population can be tested for the H64 and/or P98 allele, and the results can be compared to known disease rates to confirm that presence of the H64 and/or P98 allele is an indicator of the propensity to develop cancer. Alternatively, for populations in which there is no known disease rate, the percentage of the sample population having the H64 and/or P98 allele can be used to extrapolate a disease rate.

The present invention provides for a method of providing prophylactic cancer treatment, including the steps of detecting the presence of an H64 and/or P98 allele of galectin-3 from a patient's serum sample, predicting that the patient has the propensity to develop cancer based on the presence of the H64 and/or P98 allele, and recommending and providing prophylactic treatment to the patient to reduce their risk of developing cancer. This method can be performed as above by any medical personnel in a hospital, medical clinic, or outpatient setting with the diagnostic kit. Samples of the patient's serum are taken and compared to control nucleic material (such as DNA, RNA, protein) in order to test for the presence of the H64 and/or P98 allele. If the results of the test show that the patient is a carrier of the H64 and/or P98 allele, the medical personnel can inform the patient that they are at risk for developing cancer and Prophylactic treatment can be sought. The prophylactic treatment can be preventative surgery, preventative drug treatment, or a combination thereof. For example, a patient who is determined to be homozygous for the H64 and/or P98 allele can elect to have preventative surgery removing tissue, such as removal of breast tissue, in order to reduce their risk of developing cancer in the future. Also, the prophylactic treatment can include lifestyle changes, such as exercise and diet. Either the presence of the homozygous or heterozygous H64 and/or P98 allele is detected, as either the homozygous or heterozygous allele can indicate the propensity to develop cancer. Again, this method can be used to predict the propensity to develop any cancer, including, but not limited to, breast cancer.

The present invention also provides for a prophylactic anticancer treatment of an effective amount of a compound restricting access to a cleavage site of an H64 allele or P98 allele of galectin-3 in a pharmaceutically acceptable carrier. More preferably, the compound restricts access to an Ala62-Tyr63 cleavage site of an H64 allele of galectin-3 in a pharmaceutically acceptable carrier. Specifically, the compound targets the cleavage site of galectin-3 for MMP-2 and MMP-9 to prohibit cleavage in the H64 allele to prohibit the onset of cancer. The compounds can also target the cleavage site prohibiting cleavage of the P98 allele. This targeting by the compound can be accomplished by any number of ways, such as, but not limited to, sterically blocking the alleles by binding to the protein or blocking MMPs activity to prohibit cleavage. Effectively, the compound allows the H64 allele to mimic the P64 allele that has a reduced propensity to become cancerous. Any compound that can perform the required targeting functions effectively can be used. The compound is provided in a pharmaceutically acceptable carrier and can be administered in various ways as further discussed below.

The compound used as a prophylactic anticancer treatment of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

Finally, the present invention provides for a method of preventing the onset or further proliferation of cancer, including the steps of detecting the presence of an H64 allele of galectin-3 from a patient's serum sample, predicting that the patient has the propensity to develop cancer based on the presence of the H64 allele, administering the prophylactic anticancer treatment described above to the patient, and restricting access to an Ala62-Tyr69 cleavage site of H64 allele of galectin-3 in the patient. This method can be performed as above by any medical personnel in a hospital, medical clinic, or outpatient setting with the diagnostic kit. Samples of the patient's serum are taken and compared to control serum in order to test for the presence of the H64 allele. If the results of the test show that the patient is a carrier of the H64 allele, the medical personnel can inform the patient that they are at risk for developing cancer and prophylactic treatment can be sought. Specifically, the prophylactic treatment can include the anticancer treatment of a compound that restricts access to an Ala62-Tyr69 cleavage site as described above. The administration of the compound can also be combined with other prophylactic treatments, such as preventative surgery and lifestyle changes. Knowledge that the H64 allele is a biomarker for the propensity to develop cancer allows for preventative treatment before the onset of cancer. Also, if a patient has already been diagnosed with cancer, detecting the presence of an H64 allele can target therapy to effectively prevent the further proliferation of the cancer and enhance chances of survival. This method can also be performed in the same manner with restricting cleavage of the P98 allele.

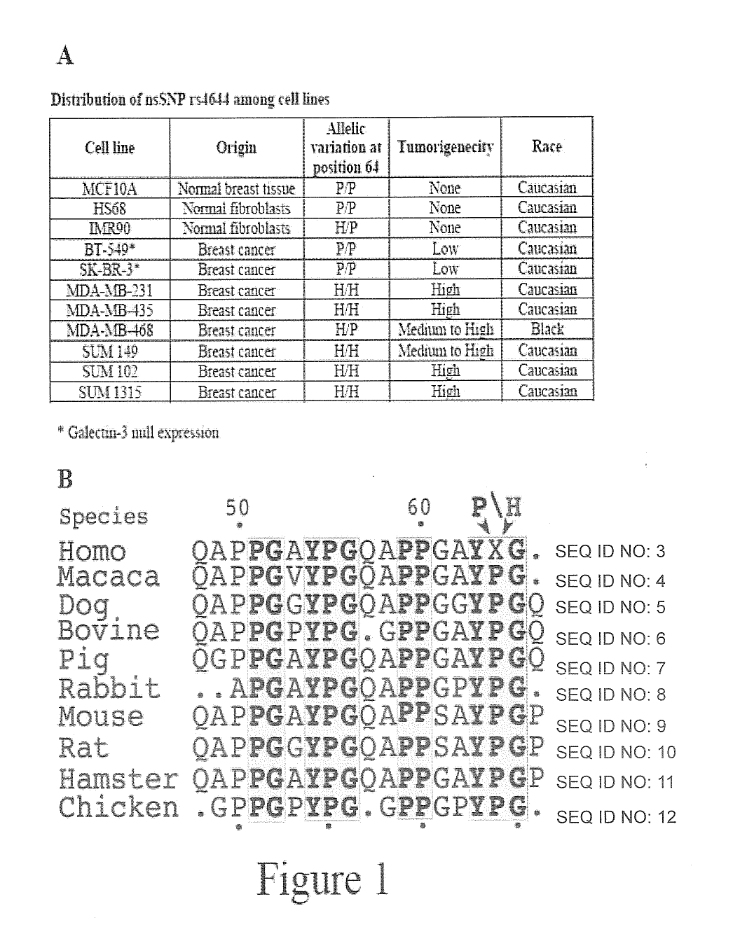

That the H64 allele is a biomarker for the propensity to develop cancer is an important discovery and is further described herein. To determine the possible significance of the nsSNP leading to P64-H64 substitution to cancer, a panel of commonly used human breast cancer cell lines were screened, their galectin-3 genomic sequences were analyzed, and then compared with their reported experimental tumorigenicity in nude mice. The normal human breast epithelial cell line (MCF10A) was found to be homozygous for the P allele similar to normal human fibroblast cells (HS68 and IMR90). Two breast carcinoma cell lines BT-549 and SK-BR-3 that are galectin-3 null and exhibited none to very low tumorigenicity in experimental models were homozygous for P allele at DNA level, while the tumorigenic human breast carcinoma cell lines, MDA-MB-231, MDA-MB-435, SUM 149, SUM 1315, and SUM 102, were homozygous for the H allele and the MDA-MB-468 was heterozygous carrying both alleles (FIG. 1A). Thus, it was determined whether in these cell lines galectin-3 P64 and galectin-3 H64 are associated with normal and tumor phenotypes, respectively. It was also determined whether this genotype variation is human specific or is evolutionarily conserved. A homology search of galectin-3 from chicken to mammals revealed that the appearance of the H64 variant is a relatively late occurrence in evolution and found only in humans (FIG. 1B).

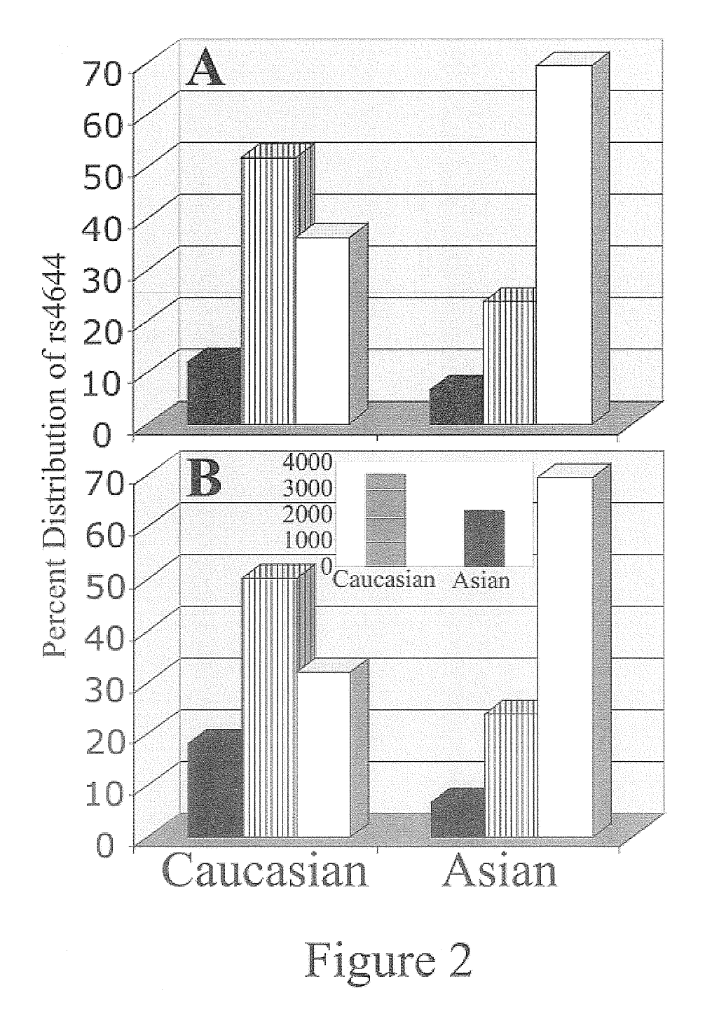

Genomic DNAs from healthy women were isolated from two control studies: one, random digit dialing; the other, volunteers. The data independently obtained from sequencing and RFLP analysis revealed that 64% of the cancer-free Caucasian women were either homozygous (12%) or heterozygous (52%) for H64 and the remaining 36% were homozygous for P64. In contrast, 70% of the cancer-free control Asian women were homozygous for P64, the remaining 30% carried at least one H64 allele, 5% of which were homozygous and 25% were heterozygous (FIG. 2A). This racial difference of genotype distribution is in accordance with the data retrieved from available databases (Ensembl release 50) (FIG. 2B).

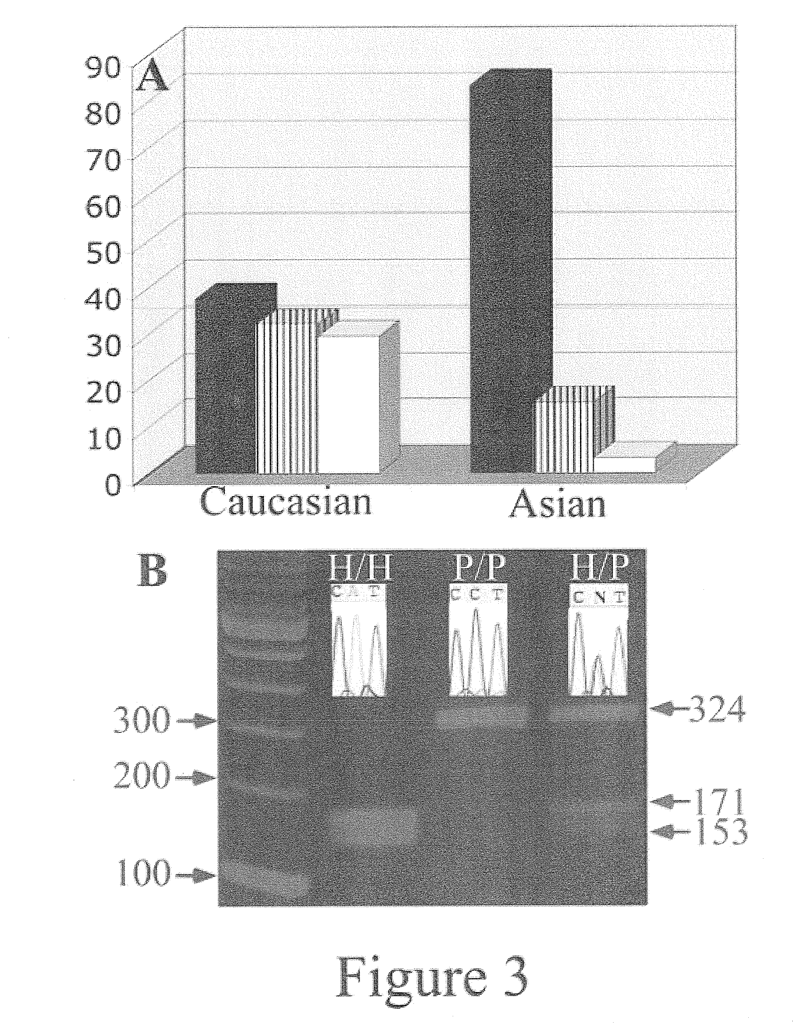

The National Cancer Institute SEER data indicated that Caucasian women have approximately 1.5 times higher propensity to develop breast cancer as compared to Asian women (FIG. 2B, inset). Genotype distribution of rs4644 in Caucasian and Asian breast cancer patients showed that 70% and 97% of the tumor samples from Caucasians and Asians, respectively, harbor the H64 allele, of which 37% of the Caucasians and 82% of the Asians were homozygous (FIG. 3A) (TABLE 1).

It was recently reported (Kienan, et al.) that Asian and European ancestors shared the same population bottleneck expanding out of Africa, but that both also experienced a more recent genetic drift, which was greater in Asians. It is assumed that this genetic drift led in part to an enrichment in the Asian population of the P genotype due to evolutionary selection. Breast cancer is a complex disease, consisting of diverse structures, genetic, epigenetic variations and clinical outcomes. Genetic linkage studies have not identified a significant number of breast cancer-causing genes other than BRCA1/2. This lack of identifiable susceptibility genes suggests that breast cancer is most probably conferred by a large number of loci, each contributing in a small additive manner to the overall breast cancer risk. In addition, variation in risk for breast cancer has been attributed in part to diet, local environmental factors, and/or yet to be identified genetic predisposing factors. Thus, to date the majority of clinical cases lack identifiable risk factor(s) other than gender, age, race, and family history. The differences in the propensity to develop breast cancer are likely attributable to multiple genes/factors and the data presented herein shows that the reported H64 and P64 allelic variation contributes, in part, to the racial disparity observed between Caucasian and Asian women in the incidence of breast cancer. There are statistically significant differences in the odds of breast cancer for the H/H phenotype compared to the P/P phenotype for Caucasian and Asian patients (odds ratio, 2.7; 95% CI 1.4-4.9, P=0.001, and odds ratio, 94.6; 95% CI 10.0-892.4, P<0.001, respectively). The H and P allelic variation reported herein is a major risk factor in the incidence of breast cancer in any population. Thus, the H and P allelic variation is an important cross-race genetic marker for breast cancer.

To establish whether this tumor-specific genotype reflects a somatic or germline change and/or loss of heterozygosity, genotyping was performed in RNA and/or DNA isolated from cancer tissue, surrounding normal tissue and blood of Caucasian and Asian breast cancer patients (TABLE 2). The genotype distribution of rs4644 from these samples was analyzed using two independent methods: DNA sequencing and PCR-based RFLP (FIG. 3B). In FIG. 3B, 324-bp amplified DNA was digested with Ncol and the resulting fragments were separated by electrophoresis on a 2% agarose gel at 100 V for 30 minutes. PCR product containing the H allele can be digested by Ncol creating two fragments of 171 and 153 by length (lanes H/H and H/P). Sequencing of the galectin-3 gene around the C191A site was performed in Applied Genomics Technology Center using the sense primer (inset: DNA sequence chromotograms indicating allelic variation). The odds ratio of breast cancer for the H/H genotype compared to the P/P genotype for Caucasian and Asian patients were 2.7, 95% CI—1.4-4.9, P=0.001 and 94.6; 95% CI—10.0-892.4, P<0.001 respectively. The data illustrated that the genotype of the normal DNA as well as RNA when available (both from blood and normal tissue) matched the genotype in tumor tissue, implying that this genetic variation was germline.

Galectin-3 has been implicated with the progression and metastasis of several types of cancers by affecting cell growth, differentiation, transformation, angiogenesis, immune response and apoptosis. While most of these functions are positively influenced by galectin-3, it can act as a double-edged sword either protecting against or stimulating cell death depending on its intra-cellular or extra-cellular localization. While over-expression of galectin-3 H64 variant resulted in resistance to cisplatin-induced apoptosis in transfected breast cancer cells BT-549, secreted extra-cellular galectin-3 H64 and its cleaved products could signal apoptosis of tumor infiltrating T cells after binding to cell surface glycol-conjugate receptors through carbohydrate dependent interactions. Moreover, this apoptosis is regulated in part by a functional cross-talk between intra-cellular and extra-cellular galectins.

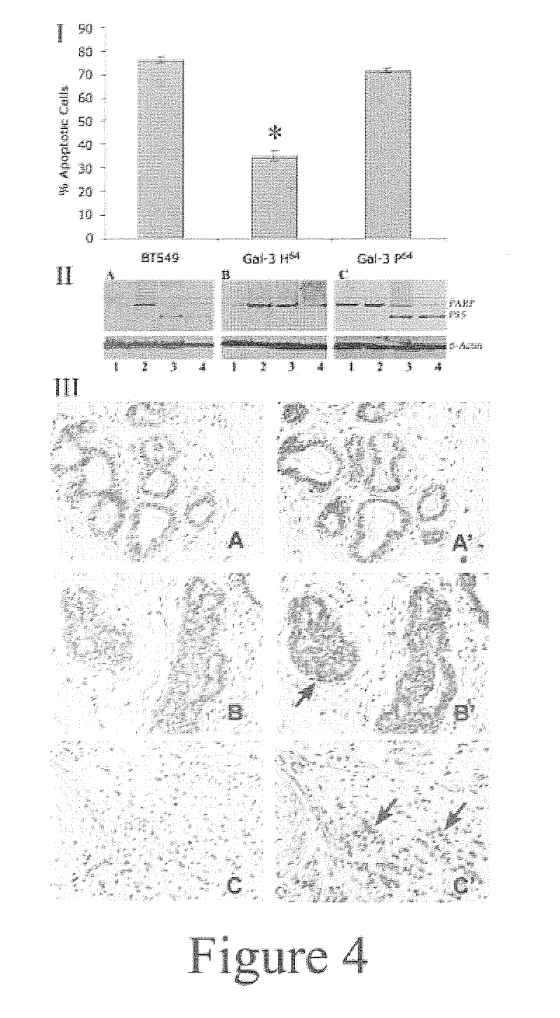

To investigate the possible functional significance of this allelic variation, BT-549 stable cell clones over-expressing either Galectin-3 P64 or H64 variant after transfection, selection, and cloning of BT-549 galectin-3 null cells were generated. The BT-549 cell clones harboring H64 and P64 variants were treated with cisplatin and a dose and time dependent decrease was observed in the number of viable cells in parental cells and P64 cell clones, whereas cells expressing the H64 variant showed a resistance to apoptosis with 36.2±2.2% apoptotic cells. In parental and P64 clone, the apoptotic cells were 76.5±1.34% and 71.5±3.02% respectively (FIG. 4I). That the cells' death was due to apoptosis was confirmed by the cleavage of PARP. FIGS. 4I-4III indicate that parental and galectin-3 P64 expressing cells showed the 85 kDa cleaved polypeptide of PARP, while galectin-3 H64 cells were refractive to PARP cleavage. FIG. 4II shows a Western blot analysis of the expression of PARP in cells treated with 50 μM cisplatin after 24, 48, and 72 hours, IIA: BT-549; IIB: BT-549 Gal-3 H64; IIC: BT-549 Gal-3 P64 (β-Actin was used as loading control. 1—untreated; 2-—24 hours; 3—48 hours; 4-—72 hours). FIG. 4III shows an immunohistochemical analysis of intact and cleaved galectin-3 in breast cancer progression, A-C: intact protein was visualized using monoclonal anti-galectin-3 antibody TIB166, A′-C′: full length plus cleaved galectin-3 using polyclonal galectin-3 antibody hL31. A-A′: normal breast tissue, B-B′: ductal hyperplasia, C-C′: infiltrating lobular carcinoma. Arrows in B′ and C′ indicate the cleaved protein, representative pictures are presented from a study of 5 normal breast tissues, 10 lobular hyperplasia, and 8 infiltrating lobular carcinoma cases.

The biological functions regulated by intracellular galectin-3 include mRNA splicing, cell growth, cell cycle and apoptosis resistance, while extra-cellular intact galectin-3/cleaved galectin-3 modulates cellular adhesion and signaling, immune response, angiogenesis and tumorigenesis by binding to cell surface glycoproteins such as laminin, fibronectin, and collagen IV. The functions regulated extra-cellularly such as chemotaxis, chemo-invasion, angiogenesis and tumor growth were all specially inhibited in the BT-549 cell clones harboring galectin-3 P64 variant. Since protein expressed by both the alleles was localized on the cell surface, it is possible that the ability of H64 variant to be cleaved by MMPs is a major contributor to the tumor progression.

A significant role of galectin-3 has been reported in the progression of breast cancer. It was suggested that a localized expression of galectin-3 in cells proximal to the stroma in comedo-DCIS could lead to increased invasive potential by inducing novel or better interactions with the stromal counterparts. This observation was supported by the over-expression of galectin-3 in invasive cell clusters and surrounding stroma, that secreted galectin-3 H64 binds to endothelial cell surface and induces its migration, morphogenesis and angiogenesis, and cleaved galectin-3 H64 bound to the endothelial cell surface with an approximately 20 times higher affinity than the full-length protein in a carbohydrate-dependent manner. Galectin-3 P64 is neither secreted nor cleaved and is unable to mimic galectin-3 H64 functions. Based on the results, not all women harboring the H allele will succumb to breast cancer, since galectin-3 is not an oncogene but awaits cancer initiation and promotion by other genes/factors and only then emancipates the transformed cells to grow and progress.

The above demonstrates data implicating galectin-3 in breast cancer development and illuminates in part, the racial disparity in the disease incidence observed between Caucasian and Asian women, and shows that the use of galectin-3 nsSNP analysis aids in conjunction with mammogram to an early diagnosis and prognosis of breast cancer.

The invention is further described in detail by reference to the following experimental examples. These examples are provided for the purpose of illustration only, and are not intended to be limiting unless otherwise specified. Thus, the present invention should in no way be construed as being limited to the following examples, but rather, be construed to encompass any and all variations which become evident as a result of the teaching provided herein.

### EXAMPLES

Materials and Methods

General Methods in Molecular Biology

Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990).

General Methods in Immunology

Standard methods in immunology known in the art and not specifically described are generally followed as in Stites et al. (eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

Immunoassays

In general, ELISAs are the preferred immunoassays employed to assess a specimen. ELISA assays are well known to those skilled in the art. Both polyclonal and monoclonal antibodies can be used in the assays. Where appropriate other immunoassays, such as radioimmunoassays (RIA) can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, Molecular Cloning: A Laboratory Manual, Cold Springs Harbor, N.Y., 1989.

Antibody Production

Antibody Production: Antibodies may be either monoclonal, polyclonal or recombinant. Conveniently, the antibodies may be prepared against the immunogen or portion thereof for example a synthetic peptide based on the sequence, or prepared recombinantly by cloning techniques or the natural gene product and/or portions thereof may be isolated and used as the immunogen. Immunogens can be used to produce antibodies by standard antibody production technology well known to those skilled in the art as described generally in Harlow and Lane, Antibodies: A Laboratory Manual, Cold Spring Harbor Laboratory, Cold Spring Harbor, N.Y., 1988 and Borrebaeck, Antibody Engineering—A Practical Guide, W.H. Freeman and Co., 1992. Antibody fragments may also be prepared from the antibodies and include Fab, F(ab′)2, and Fv by methods known to those skilled in the art.

For producing polyclonal antibodies a host, such as a rabbit or goat, is immunized with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific.

For producing monoclonal antibodies the technique involves hyperimmunization of an appropriate donor with the immunogen, generally a mouse, and isolation of splenic antibody producing cells. These cells are fused to a cell having immortality, such as a myeloma cell, to provide a fused cell hybrid that has immortality and secretes the required antibody. The cells are then cultured, in bulk, and the monoclonal antibodies harvested from the culture media for use.

For producing recombinant antibody (see generally Huston et al, 1991; Johnson and Bird, 1991; Mernaugh and Mernaugh, 1995), messenger RNAs from antibody producing B-lymphocytes of animals, or hybridoma are reverse-transcribed to obtain complimentary DNAs (cDNAs). Antibody cDNA, which can be full or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated as is well known in the art. (For a general discussion of conjugation of fluorescent or enzymatic moieties see Johnstone & Thorpe, Immunochemistry in Practice, Blackwell Scientific Publications, Oxford, 1982.) The binding of antibodies to a solid support substrate is also well known in the art. (see for a general discussion Harlow & Lane Antibodies: A Laboratory Manual, Cold Spring Harbor Laboratory Publications, New York, 1988 and Borrebaeck, Antibody Engineering—A Practical Guide, W.H. Freeman and Co., 1992) The detectable moieties contemplated with the present invention can include, but are not limited to, fluorescent, metallic, enzymatic and radioactive markers such as biotin, gold, ferritin, alkaline phosphatase, b-galactosidase, peroxidase, urease, fluorescein, rhodamine, tritium, 14C and iodination.

Control and Patients Population

The racial distribution of nsSNP rs4644 in normal population was retrieved from Ensembl release 50 (50 Caucasian and 59 Asian), blood DNA samples from randomly selected women from the Southeast Michigan Tri-county area (139 Caucasian) and an additional 25 disease free volunteers (5 Caucasian and 20 Asian). DNA samples from the breast cancer patients were isolated from the frozen tissues of 43 Caucasian and 45 Asian (Asterand, Detroit, Mich.) and 71 Caucasian and 1 Asian women (Southeast Michigan Tri-county area). Matched DNA (from blood, frozen normal, and cancer) from 15 Asian breast cancer patients was obtained from the Medical School of Zhengzhou University, 10 Caucasian matched frozen cancer and normal breast DNA were obtained from Asterand. Matched DNA from frozen cancer, normal tissue, and blood, and RNA from cancer and normal tissue from 17 Caucasian patients were obtained from the Biospecimens Core of the Mayo Breast Cancer SPORE (Rochester, Minn.). The mean age of the combined breast cancer group was 51.7 years. In all cases, the diagnosis was pathologically confirmed. DNA was isolated using ACCUPREP® Genomic DNA extraction kit (Bioneer, Alameda, Calif.).

PCR and Sequencing

The following primer pair was designed to amplify exon 3 of human galectin-3:

SEQ ID NO: 1
Sense 5′ CTCCATGATGCGTTATCTGGGTCTGG 3′
SEQ ID NO: 2
Anti-sense 3′ CCTATGGCGCCCCTGCTGGGCCACTG 5′

The target sequence was amplified using EPPENDORF® Thermal Cycler (Qiagen) in a 20 μl reaction volume containing 100 ng of genomic DNA, 0.5 μM of each primer, 10 μl of QIAGEN Fast Cycling PCR Master Mix and 4 μl of Q-Solution. Amplification conditions were an initial activation step of 95° C. for 5 minutes followed by 35 cycles of denaturation at 96° C. for 5 seconds, annealing at 60° C. for 5 seconds, and extension at 68° C. for 9 seconds. The resulting 324 by DNA fragment was purified using QIAQUICK® PCR Purification Kit (Qiagen, Valencia, Calif.), visualized on 1% agarose gel, containing 100 nM of SYTO® 60 stain, scanned by Odyssey infrared imaging system (LI-COR Biosciences, Lincoln, Nebr.) to confirm the size and purity and sequenced using the sense primer at Applied Genomics Technology Center, Core facility at Wayne State University and Karmanos Cancer Institute. PCR-based RFLP was performed by digestion of the PCR product with Ncol (Invitrogen Corporation, Carlsbad, Calif.) as an additional method to confirm the genotype. Both experiments were performed in a blinded manner by two investigators independently on two different dates. To verify that the DNA samples were not cross-contaminated, the entire length of each PCR product was sequenced and distribution of a different nsSNP in exon 3 was observed, implying no cross-contamination.

Cell Lines and Antibodies

The human breast cancer cell lines SK-BR3, MDA-MB-435, MDA-MB-231, MDA-MB-468, and normal fibroblast cells IMR-90 and HS-68, were maintained in McCoy medium (Invitrogen Corporation, Carlsbad, Calif.) supplemented with 10% fetal bovine serum (ATLANTA BIOLOGICALS, Lawrenceville, Ga.). Normal breast cell line MCF10A was maintained in DMEM F-12 medium (Invitrogen Corporation, Carlsbad, Calif.) with 0.1 μg/mL cholera toxin, 19 μg/mL insulin, 0.5 μg/mL hydrocortisone, 0.02 μg/mL epidermal growth factor, 5% horse serum (Sigma-Aldrich Inc., St. Louis, Mo.). BT-549 and its stably transfected cell clones, BT-549 Gal-3 H64, BT-549 Gal-3 P64 constructed as described (Cancer Res. In press) were maintained in Dulbecco's Minimal Essential Medium (Invitrogen Corporation, Carlsbad, Calif.) containing 10% fetal bovine serum, essential and non-essential amino acids (Invitrogen), vitamins and antibiotics (Mediatech Cellgro Inc., Herdon, Va.), SUM-102, SUM-149 and SUM 1315 were maintained in Ham's F-12 medium supplemented with 10% fetal bovine serum, 5 μg/mL insulin, 1 μg/mL hydrocortisone (Sigma-Aldrich Inc., St-Louis, Mo.). All the cells were grown in 5% CO2 incubator at 37° C. Cells were grown to 80% confluence and genomic DNA was extracted using ACCUPREP® Genomic DNA extraction kit (Bioneer, Alameda, Calif.). Anti-poly (ADP-ribose) polymerase (PARP), mouse monoclonal antibody was purchased from (BioMol, Plymouth Meeting, Pa.). Monoclonal anti-β-actin (clone AC-15) was purchased from (Sigma-Aldrich, St. Louis, Mo.). Hybridoma expressing galectin-3 monoclonal antibody TIB-166 was purchased from American Type Culture Collection (Manassas, Va.). The polyclonal antibody against galectin-3 was prepared as described.

Cell Proliferation and Viability

2500 BT-549, BT-549 Gal-3 H64 and BT-549 Gal-3 P64 cells were seeded per well in triplicates onto 24 well culture dishes. In one set of wells, 25 μM cisplatin (Sigma-Aldrich Inc., St. Louis, Mo.) was added after 24 hr of seeding, the other non-treated set served as a control. Cell viability/apoptosis was determined after 72 hours by the mitochondrial-dependent reduction of 3-(4,5-dimethylthiazol-2-yl)-2,5-diphenyltetrazolium bromide (MTT) (Sigma-Aldrich Inc., St. Louis, Mo.) to formazan.

Cisplatin Treatment and Western Blot Analysis

To determine if cell death is due to apoptosis, 1×106 cells were seeded in 100 mm Petri dishes. After 24 hours of serum starvation, the cells were incubated with 25 μM cisplatin. After 24, 48, 72 hours, the cells were trypsinized, lysed and equivalent number of cells (1×105) were subjected to SDS-PAGE and Western blot analysis with a 1:200 dilution of anti-poly (ADP-ribose) polymerase (PARP), mouse monoclonal antibody (BioMol, Plymouth Meeting, Pa.). Blots were also immuno-reacted with a 1:5000 dilution of anti-b-actin mouse monoclonal antibody to normalize for variation in protein loading. Following washes the blots were reacted with secondary antibody mix containing 1:5000 dilutions of IR Dye 680 or IR Dye 800 conjugated corresponding antibody (Molecular Probes, Eugene, Oreg.) and scanned by Odyssey infra red imaging system (LI-COR Biosciences, Lincoln, Nebr.) to identify the respective proteins.

Immunohistochemical Analysis

A breast cancer progression tissue array (BR480) containing 48 tissue cores of 2 mm size was purchased from U.S. Biomax (Rockville, Md.). Four μm serial sections were deparafinized, rehydrated, blocked, and incubated with primary antibodies (anti-galectin-3 monoclonal, anti-galectin-3 polyclonal) at 4° C. overnight at the suitable dilution and linked with the appropriate host secondary antibodies (Vector Laboratories, Burlingame, Calif.) tagged with Avidin biotinylated horseradish peroxidase, colorized with 3′-3′-diaminobenzidine and counterstained with hematoxylin. Visualization and documentation were accomplished with an OLYMPUS BX40 microscope supporting a Sony DXC-979MD 3CCCD video camera and stored with the M5+ micro-computer imaging device (Interfocus, Cambridge, UK).

Statistical Analysis

The assumption of Hardy-Weinberg equilibrium in the control groups was evaluated using Fisher exact tests. Odd ratios (ORs) were used to estimate the magnitude of the association of breast cancer with the alleles HH and HP. Cornfield's method was used to estimate 95% confidence intervals (CIs). If there was no evidence of heterogeneity in the ORs based on Tarone's test, the Mantel-Haenszel combined OR was calculated to estimate the overall OR across ethnic groups. Two-sided p-values are reported. Calculations were made with Stata SE 10.2.

The assumption of Hardy-Weinberg equilibrium was found to be tenable in both the Asian control group (p=0.12) and the Caucasian control group (p=0.44). The Biostatistics Core of the Karmanos Cancer Institute performed statistical analysis. Microsoft Excel software was used for calculating statistical significance of the apoptosis assay by 2 sample t test using unequal variance. P values of <0.05 were considered statistically significant.

In summary, the above data shows that a functional germ-line mutation in the human galectin-3 at its matrix metalloproteinase cleavage site due to a nsSNP at position 191 (rs4644) resulting in a substitution of proline with histidine (P64H) at the amino acid residue 64, was associated with more than 97% of breast cancer cases examined. Genotypic analysis of Caucasian and Asian women revealed that the difference in their rs4644 genotype distribution is reflected in the relative incidence ratio of the breast cancer incidences between the races. These results show that the H64 allele is an important biomarker for cancer and can be used as a diagnostic tool to determine both patients' and populations' propensity for developing cancer.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

TABLE 1

| | | | Allelic |
|---|---|---|---|
| Sample | | | variation at |
| ID | Race | Diagnose | position 64 |
| 10173 A1 | Asian | Invasive Ductal Carcinoma | H/H |
| 10181 A1 | Asian | Invasive Ductal Carcinoma | H/H |
| 10195 A1 | Asian | Invasive Ductal Carcinoma | H/H |
| 10207 A1 | Asian | Invasive Ductal Carcinoma | H/H |
| 10212 A1 | Asian | Invasive Ductal Carcinoma | H/H |
| 107 A1 | Asian | Invasive Ductal Carcinoma | H/H |
| 114 A1 | Asian | Invasive Ductal Carcinoma | H/H |
| 11767 A1 | Asian | Invasive small lobular carcinoma | H/H |
| 154 A1 | Asian | Infiltrating ductal carcinoma | H/H |
| 163 A1 | Asian | Interstitial Sarcoma of the breast | H/H |
| 165 A1 | Asian | Invasive ductal carcinoma | H/H |
| 170 A1 | Asian | Invasive ductal carcinoma | H/H |
| 176 A1 | Asian | Invasive ductal carcinoma | H/H |
| 267 A1 | Asian | Invasive ductal carcinoma | H/H |
| 311 A1 | Asian | Invasive ductal carcinoma | H/H |
| 342 A1 | Asian | Comedocarcinoma | H/H |
| 3489 A1 | Asian | Infiltrating ductal carcinoma | H/H |
| 3490 A1 | Asian | Infiltrating ductal carcinoma | H/H |
| 3495 A1 | Asian | Invasive ductal carcinoma | H/H |
| 3512 A1 | Asian | Invasive small lobe carcinoma | H/H |
| 3569 A1 | Asian | Carcinoma simplex of breast | H/H |
| 3631 A1 | Asian | Carcinoma simplex of breast | H/H |
| 3635 A1 | Asian | Breast cancer | H/H |
| 3811 A1 | Asian | Breast cancer | H/H |
| 3821 A1 | Asian | Breast cancer | H/H |
| 3822 A1 | Asian | Invasive ductal carcinoma | H/H |
| 3831 A1 | Asian | Invasive ductal carcinoma | H/H |
| 3871 B1 | Asian | Simplex carcinoma | H/H |
| 3900 A1 | Asian | Carcinoma simplex of breast | H/H |
| 4241 A1 | Asian | Breast cancer | H/H |
| 452 A1 | Asian | Breast cancer | H/H |
| 4703 A1 | Asian | Invasive ductal carcinoma | H/H |
| 6873 A1 | Asian | Breast cancer | H/H |
| 6879 A1 | Asian | Invasive ductal carcinoma | H/H |
| 6884 A1 | Asian | Invasive ductal carcinoma | H/H |
| 7350 B1 | Asian | Carcinoma simplex | H/H |
| 7746 B1 | Asian | Scirrhous carcinoma | H/H |
| 9361 A1 | Asian | Infiltrating ductal carcinoma | H/H |
| 9578 A1 | Asian | Invasive ductal carcinoma | H/H |
| 9579 A1 | Asian | Infiltrating ductal carcinoma | H/H |
| 9611 A1 | Asian | Invasive ductal carcinoma | H/H |
| 9626 A1 | Asian | Invasive ductal carcinoma | H/H |
| 9776 B1 | Asian | Invasive ductal carcinoma | H/H |
| 22B | Asian | Invasive ductal carcinoma | H/P |
| 343 | Asian | Invasive ductal carcinoma | H/P |
| 9612 | Asian | Invasive ductal carcinoma | H/P |
| 4678 | Asian | Invasive ductal carcinoma | H/P |
| 10 | Asian | Invasive ductal carcinoma | H/P |
| 3866 | Asian | Invasive ductal carcinoma | P/P |
| 10201 | Asian | Invasive ductal carcinoma | P/P |
| 10167 A1 | Asian | Invasive ductal carcinoma | P/P |
| 10172 A1 | Asian | Invasive ductal carcinoma | P/P |
| 3823 | Asian | Invasive ductal carcinoma | P/P |
| 4899 | Caucasian | Lobular carcinoma | H/H |
| 7181 | Caucasian | Ductal carcinoma | H/H |
| 11466 B1 | Caucasian | Ductal carcinoma | H/H |
| 11678 A1 | Caucasian | Infiltrating duct breast carcinoma | H/H |
| 14894 A1 | Caucasian | Lobular carcinoma | H/H |
| 1731 B1 | Caucasian | Infiltrating ductal carcinoma | H/H |
| 1772 A1 | Caucasian | Infiltrating ductal carcinoma | H/H |
| 1778 B1 | Caucasian | Infiltrating ductal carcinoma | H/H |
| 199 A1 | Caucasian | Infiltrating ductal carcinoma | H/H |
| 336 A1 | Caucasian | Infiltrating ductal carcinoma | H/H |
| 4047 A1 | Caucasian | Infiltrating ductal carcinoma | H/H |
| 4526 B1 | Caucasian | Lobular carcinoma | H/H |
| 4541 B1 | Caucasian | Lobular carcinoma | H/H |
| 4607 A1 | Caucasian | Infiltrating ductal carcinoma | H/H |
| 5470 B1 | Caucasian | Lobular carcinoma | H/H |
| 5521 B1 | Caucasian | Infiltrating ductal carcinoma | H/H |
| 5860 A1 | Caucasian | Infiltrating ductal carcinoma | H/H |
| 6617 A1 | Caucasian | Lobular breast carcinoma | H/H |
| 6660 A1 | Caucasian | Infiltrating ductal carcinoma | H/H |
| 6699 B1 | Caucasian | Infiltrating lobular carcinoma | H/H |
| 6711 A1 | Caucasian | Infiltrating ductal carcinoma | H/H |
| 6713 A1 | Caucasian | Infiltrating ductal carcinoma | H/H |
| 6716 A1 | Caucasian | Lobular carcinoma | H/H |
| 6731 A1 | Caucasian | Infiltrating ductal carcinoma | H/H |
| 69 A1 | Caucasian | Infiltrating ductal carcinoma | H/H |
| 7328 B1 | Caucasian | Lobular carcinoma | H/H |
| 7331 B1 | Caucasian | Infiltrating duct carcinoma | H/H |
| 7354 B1 | Caucasian | Lobular carcinoma | H/H |
| 7376 B1 | Caucasian | Lobular carcinoma | H/H |
| 7391 B1 | Caucasian | Infiltrating ductal carcinoma | H/H |
| 7409 B1 | Caucasian | Lobular carcinoma | H/H |
| 7411 B1 | Caucasian | Lobular carcinoma | H/H |
| 7415 B1 | Caucasian | Infiltrating Ductal Carcinoma | H/H |
| 8276 A1 | Caucasian | Infiltrating Ductal Carcinoma | H/H |
| 8301 A1 | Caucasian | Infiltrating Ductal Carcinoma | H/H |
| 9293 A1 | Caucasian | Infiltrating Ductal Carcinoma | H/H |
| 9307 A1 | Caucasian | Infiltrating Ductal Carcinoma | H/H |
| 9329 A1 | Caucasian | Infiltrating Ductal Carcinoma | H/H |
| 9352 A1 | Caucasian | Infiltrating Ductal Carcinoma | H/H |
| 9390 A1 | Caucasian | Infiltrating Ductal Carcinoma | H/H |
| 9895 B1 | Caucasian | Infiltrating Ductal Carcinoma | H/H |
| 12 | Caucasian | Lobular carcinoma | H/H |
| 14 | Caucasian | Infiltrating Ductal Carcinoma | H/H |
| 42 | Caucasian | Infiltrating Ductal Carcinoma | H/H |
| 51 | Caucasian | Infiltrating Ductal Carcinoma | H/H |
| 60 | Caucasian | Infiltrating Ductal Carcinoma | H/H |
| 85 | Caucasian | Infiltrating Ductal Carcinoma | H/H |
| 91 | Caucasian | Infiltrating Ductal Carcinoma | H/H |
| 1AD | Caucasian | Infiltrating Ductal Carcinoma | H/H |
| 20AD | Caucasian | Infiltrating Ductal Carcinoma | H/H |
| 3AD | Caucasian | Infiltrating Ductal Carcinoma | H/H |
| 5AD | Caucasian | Infiltrating Ductal Carcinoma | H/H |
| 7AD | Caucasian | Infiltrating Ductal Carcinoma | H/H |
| 4067 A1 | Caucasian | Breast cancer | H/H |
| 4108 A1 | Caucasian | Infiltrating Ductal Carcinoma | H/H |
| 4153 A1 | Caucasian | Infiltrating Ductal Carcinoma | H/P |
| 83 | Caucasian | Breast cancer | H/P |
| 4069 | Caucasian | Infiltrating Ductal Carcinoma | H/P |
| 6713 | Caucasian | Infiltrating Ductal Carcinoma | H/P |
| 7852 | Caucasian | Infiltrating Ductal Carcinoma | H/P |
| 9394 | Caucasian | Infiltrating Ductal Carcinoma | H/P |
| 4 | Caucasian | Breast cancer | H/P |
| 5 | Caucasian | Breast cancer | H/P |
| 6 | Caucasian | Breast cancer | H/P |
| 9 | Caucasian | Breast cancer | H/P |
| 15 | Caucasian | Breast cancer | H/P |
| 19 | Caucasian | Breast cancer | H/P |
| 29 | Caucasian | Breast cancer | H/P |
| 36 | Caucasian | Breast cancer | H/P |
| 41 | Caucasian | Breast cancer | H/P |
| 43 | Caucasian | Breast cancer | H/P |
| 44 | Caucasian | Breast cancer | H/P |
| 52 | Caucasian | Breast cancer | H/P |
| 56 | Caucasian | Breast cancer | H/P |
| 57 | Caucasian | Breast cancer | H/P |
| 59 | Caucasian | Breast cancer | H/P |
| 61 | Caucasian | Breast cancer | H/P |
| 62 | Caucasian | Breast cancer | H/P |
| 64 | Caucasian | Breast cancer | H/P |
| 70 | Caucasian | Breast cancer | H/P |
| 71 | Caucasian | Breast cancer | H/P |
| 75 | Caucasian | Breast cancer | H/P |
| 80 | Caucasian | Breast cancer | H/P |
| 81 | Caucasian | Breast cancer | H/P |
| 86 | Caucasian | Breast cancer | H/P |
| 87 | Caucasian | Breast cancer | H/P |
| 89 | Caucasian | Breast cancer | H/P |
| 90 | Caucasian | Breast cancer | H/P |
| 94 | Caucasian | Breast cancer | H/P |
| 99 | Caucasian | Breast cancer | H/P |
| 100 | Caucasian | Breast cancer | H/P |
| 18AD | Caucasian | Infiltrating Ductal Carcinoma | H/P |
| 10AD | Caucasian | Infiltrating Ductal Carcinoma | H/P |
| 11AD | Caucasian | Infiltrating Ductal Carcinoma | H/P |
| 12AD | Caucasian | Infiltrating Ductal Carcinoma | H/P |
| 15AD | Caucasian | Infiltrating Ductal Carcinoma | H/P |
| 17AD | Caucasian | Infiltrating Ductal Carcinoma | H/P |
| 18AD | Caucasian | Infiltrating Ductal Carcinoma | H/P |
| 19AD | Caucasian | Infiltrating Ductal Carcinoma | H/P |
| 4AD | Caucasian | Infiltrating Ductal Carcinoma | H/P |
| 9AD | Caucasian | Infiltrating Ductal Carcinoma | H/P |
| 2633 | Caucasian | Infiltrating ductal carcinoma | H/P |
| 3133 | Caucasian | Infiltrating ductal carcinoma | H/P |
| 3879 | Caucasian | Infiltrating ductal carcinoma | H/P |
| 4097 | Caucasian | Lobular carcinoma | H/P |
| 4235 | Caucasian | Lobular carcinoma | H/P |
| 5041 | Caucasian | Infiltrating ductal carcinoma | H/P |
| 14067 B1 | Caucasian | Lobular carcinoma | H/P |
| 17370 B1 | Caucasian | Infiltrating ductal carcinoma | H/P |
| 40869 A1 | Caucasian | Infiltrating ductal carcinoma | H/P |
| 40979 B1 | Caucasian | Lobular breast carcinoma | H/P |
| 4465 A1 | Caucasian | Infiltrating ductal carcinoma | H/P |
| 5550 B1 | Caucasian | Infiltrating lobular carcinoma | H/P |
| 6661 B1 | Caucasian | Infiltrating ductal carcinoma | H/P |
| 6712 A1 | Caucasian | Infiltrating ductal carcinoma | H/P |
| 9185 B1 | Caucasian | Lobular carcinoma | H/P |
| 9904 B1 | Caucasian | Infiltrating ductal carcinoma | H/P |
| 2943 | Caucasian | Infiltrating ductal carcinoma | P/P |
| 39 | Caucasian | Breast cancer | P/P |
| 4123 | Caucasian | Infiltrating ductal carcinoma | P/P |
| 6AD | Caucasian | Infiltrating ductal carcinoma | P/P |
| 2974 | Caucasian | Infiltrating ductal carcinoma | P/P |
| 3556 | Caucasian | Infiltrating ductal carcinoma | P/P |
| 4496 | Caucasian | Infiltrating ductal carcinoma | P/P |
| 4566 | Caucasian | Infiltrating ductal carcinoma | P/P |
| 4870 | Caucasian | Infiltrating ductal carcinoma | P/P |
| 7092 | Caucasian | Infiltrating ductal carcinoma | P/P |
| 7145 | Caucasian | Infiltrating ductal carcinoma | P/P |
| 4472 A1 | Caucasian | Infiltrating ductal carcinoma | P/P |
| 9892 | Caucasian | Infiltrating ductal carcinoma | P/P |
| 1 | Caucasian | Breast cancer | P/P |
| 3 | Caucasian | Breast cancer | P/P |
| 7 | Caucasian | Breast cancer | P/P |
| 8 | Caucasian | Breast cancer | P/P |
| 11 | Caucasian | Breast cancer | P/P |
| 20 | Caucasian | Breast cancer | P/P |
| 22 | Caucasian | Breast cancer | P/P |
| 23 | Caucasian | Breast cancer | P/P |
| 26 | Caucasian | Breast cancer | P/P |
| 27 | Caucasian | Breast cancer | P/P |
| 30 | Caucasian | Breast cancer | P/P |
| 32 | Caucasian | Breast cancer | P/P |
| 34 | Caucasian | Breast cancer | P/P |
| 37 | Caucasian | Breast cancer | P/P |
| 38 | Caucasian | Breast cancer | P/P |
| 40 | Caucasian | Breast cancer | P/P |
| 47 | Caucasian | Breast cancer | P/P |
| 48 | Caucasian | Breast cancer | P/P |
| 49 | Caucasian | Breast cancer | P/P |
| 54 | Caucasian | Breast cancer | P/P |
| 55 | Caucasian | Breast cancer | P/P |
| 58 | Caucasian | Breast cancer | P/P |
| 67 | Caucasian | Breast cancer | P/P |
| 69 | Caucasian | Breast cancer | P/P |
| 73 | Caucasian | Breast cancer | P/P |
| 74 | Caucasian | Breast cancer | P/P |
| 81 | Caucasian | Breast cancer | P/P |
| 84 | Caucasian | Breast cancer | P/P |
| 95 | Caucasian | Breast cancer | P/P |
| 96 | Caucasian | Breast cancer | P/P |
| 97 | Caucasian | Breast cancer | P/P |
| 98 | Caucasian | Breast cancer | P/P |
| 13AD | Caucasian | Infiltrating ductal carcinoma | P/P |
| 14AD | Caucasian | Infiltrating ductal carcinoma | P/P |
| 16AD | Caucasian | Infiltrating ductal carcinoma | P/P |
| 8AD | Caucasian | Infiltrating ductal carcinoma | P/P |
| 2AD | Caucasian | Infiltrating ductal carcinoma | P/P |

TABLE 2

| | DNA | DNA | DNA | RNA | RNA |
|---|---|---|---|---|---|
| Race | Blood | Normal | Cancer | cancer | Normal |
| Caucasian | NA | H/P | H/P | NA | NA |
| Caucasian | NA | H | H | NA | NA |
| Caucasian | NA | H/P | H/P | NA | NA |
| Caucasian | NA | H | H | NA | NA |
| Caucasian | NA | H/P | H/P | NA | NA |
| Caucasian | NA | H | H | NA | NA |
| Caucasian | NA | H/P | H/P | NA | NA |
| Caucasian | NA | H | H | NA | NA |
| Caucasian | NA | H/P | H/P | NA | NA |
| Caucasian | NA | H | H | NA | NA |
| Caucasian | H/H | H/H | H/H | H/H | H/H |
| Caucasian | P/P | P/P | P/P | P/P | P/P |
| Caucasian | H/H | H/H | H/H | H/H | NA |
| Caucasian | H/H | H/H | H/H | H/H | H/H |
| Caucasian | H/H | H/H | H/H | H/H | H/H |
| Caucasian | P/P | P/P | P/P | P/P | NA |
| Caucasian | H/P | H/P | H/P | H/P | NA |
| Caucasian | H/P | H/P | H/P | H/P | H/P |
| Caucasian | H/P | H/P | H/P | H/P | H/P |
| Caucasian | P/P | P/P | P/P | NA | NA |
| Caucasian | P/P | P/P | P/P | P/P | P/P |
| Caucasian | H/P | H/P | H/P | H/P | H/P |
| Caucasian | P/P | P/P | P/P | P/P | NA |
| Caucasian | H/P | H/P | H/P | H/P | H/P |
| Caucasian | H/P | H/P | H/P | H/P | H/P |
| Caucasian | H/P | H/P | H/P | H/P | H/P |
| Caucasian | H/H | H/H | H/H | H/H | H/H |
| Asian | H/P | H/P | H/P | NA | NA |
| Asian | H | H | H | NA | NA |
| Asian | H/P | H/P | H/P | NA | NA |
| Asian | H/P | H/P | H/P | NA | NA |
| Asian | H | H | H | NA | NA |
| Asian | H | H | H | NA | NA |
| Asian | H | H | H | NA | NA |
| Asian | H | H | H | NA | NA |
| Asian | H | H | H | NA | NA |
| Asian | H/P | H/P | H/P | NA | NA |
| Asian | H | H | H | NA | NA |
| Asian | H/P | H/P | H/P | NA | NA |
| Asian | H/P | H/P | H/P | NA | NA |
| Asian | H/P | H/P | H/P | NA | NA |
| Asian | H/P | H/P | H/P | NA | NA |

NA = Not Available

What is claimed is:

1. A method of predicting propensity to develop cancer, including the steps of:
detecting the presence of an allele of galectin-3 chosen from the group consisting of a homozygous H64 allele, a heterozygous H64 allele, a homozygous P98 allele, a heterozygous P98 allele, and combinations thereof from a patient's serum sample; and
predicting that the patient has the propensity to develop cancer based on the presence of the allele.

2. The method of claim 1, wherein the cancer is breast cancer.

3. The method of claim 1, wherein the detecting step is further defined as detecting the allele by analyzing a component of the patient's serum chosen from the group consisting of DNA, mRNA, and protein.

4. A method of predicting a population's propensity to develop cancer, including the steps of:
detecting the presence of an allele of galectin-3 chosen from the group consisting of a homozygous H64 allele, a heterozygous H64 allele, a homozygous P98 allele, a heterozygous P98 allele, and combinations thereof from serum samples of patients in a population; and
predicting the percentage of the population that has the propensity to develop cancer based on the presence of the allele.

5. The method of claim 4, wherein the cancer is breast cancer.

6. The method of claim 4, wherein the detecting step is further defined as detecting the allele by analyzing a component of the patient's serum chosen from the group consisting of DNA, mRNA, and protein.

7. A method of providing prophylactic cancer treatment, including the steps of:
detecting the presence of an allele of galectin-3 chosen from the group consisting of a homozygous H64 allele, a heterozygous H64 allele, a homozygous P98 allele, a heterozygous P98 allele, and combinations thereof from a patient's serum sample;
predicting that the patient has the propensity to develop cancer based on the presence of the allele; and
recommending and providing prophylactic treatment to the patient to reduce their risk of cancer.

8. The method of claim 7, wherein said prophylactic treatment is chosen from the group consisting of preventative surgery, preventative drug treatment, and combinations thereof.

9. The method of claim 7, wherein the cancer is breast cancer. The method of claim 7, wherein the detecting step is further defined as detecting the allele by analyzing a component of the patient's serum chosen from the group consisting of DNA, mRNA, and protein.
